(19) United States
(12) Patent Application Publication
(10) Pub. No.: US 2025/0017956 A1
(43) Pub. Date: Jan. 16, 2025

(54) CROSSLINKED HYDROGELS WITH ENHANCED RADIOPACITY FOR MEDICAL APPLICATIONS

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US); Rick Dorn, Plymouth, MN (US); Cristian Parisi, Boston, MA (US); Niraj Gurung, Sauk Rapids, MN (US); Claire Hobbs, Cork (IE)

(73) Assignee: Boston Scientific Scimed, Inc., Maple Grove, MN (US)

(21) Appl. No.: 18/762,216

(22) Filed: Jul. 2, 2024

Related U.S. Application Data
(60) Provisional application No. 63/525,223, filed on Jul. 6, 2023.

(51) Int. Cl. A61K 31/765; A61K 45/06; C08J 3/075; C08K 3/011

(52) U.S. Cl. CPC A61K 31/765; A61K 45/06; C08J 3/075; C08K 3/011

(57) ABSTRACT

Disclosed herein are radiopaque, reactive multi-arm polymers that comprise an iodine-containing core region, a plurality of polymer arms comprising a plurality of polymer segments linked to the iodine-containing core region, and a plurality of reactive moieties linked to the plurality of polymer segments. Also disclosed are methods of forming such radiopaque, reactive multi-arm polymers, systems for forming hydrogel compositions that comprise (a) such radiopaque, reactive multi-arm polymers and (b) multifunctional crosslinking compounds comprising a plurality of complementary reactive moieties that are reactive with the reactive moieties of the radiopaque, reactive multi-arm polymers, as well as reaction products of such systems and methods of treatment using such systems.

### CROSS REFERENCE TO RELATED APPLICATIONS

This application claims the benefit of U.S. Provisional Patent Application Ser. No. 63/525,223 filed on Jul. 6, 2023, the disclosure of which is incorporated herein by reference.

### FIELD

The present disclosure relates radiopaque hydrogels and to crosslinkable systems for forming radiopaque hydrogels, among other aspects. The radiopaque hydrogels and crosslinkable systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer functionalized with succinimidyl glutarate as activated end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications.

While the above approach is effectual, some issues arise as a result of the incorporation of the TIB functional group. First, in order to functionalize TIB on 8-arm PEG, one succinimidyl glutarate binding site is sacrificed for each TIB-functionalized arm. Moreover, the entire functionalization process involves multiple steps, typically five steps, from commercially available hydroxyl-terminated 8-arm PEG to its functionalized form with two different end groups (2,3,5-triiiodobenzamide and succinimidyl glutarate groups). This complex process of synthesizing the 8-arm PEG results in a significant increase of the product cost, decreased hydrogel persistence, and difficulties in product quality control. Furthermore, each added 2,3,5-triiiodobenzamide group occupies one arm of the star-PEG, reducing capacity and efficiency of the crosslinking reaction.

For these and other reasons, alternative strategies for forming iodine-labelled crosslinked hydrogels that provide enhanced radiopacity while maintaining crosslink density per polymer molecule are desired.

### SUMMARY

The present disclosure provides an alternative approach to that described above.

In some aspects, the present disclosure provides radiopaque, reactive multi-arm polymers that comprise an iodine-containing core region, a plurality of polymer arms comprising a plurality of polymer segments linked to the iodine-containing core region, and a plurality of reactive moieties linked to the plurality of polymer segments.

In some embodiments, radiopaque, reactive multi-arm polymer further comprises a plurality of hydrolysable ester groups positioned between the plurality of reactive moieties and the plurality of polymer segments.

In some embodiments, which can be used in conjunction with the above aspects and embodiments, the radiopaque, the plurality of polymer segments comprise one or more monomer residues selected from C₁-C₄-alkylene oxide residues, oxazoline monomer residues, cyclic ester monomer residues and propylene oxide and fumarate comonomers (that can generate degradable copolymer).

In some embodiments, which can be used in conjunction with the above aspects and embodiments, the polymer segments contain between 10 and 1000 monomer residues.

In some embodiments, which can be used in conjunction with the above aspects and embodiments, the reactive moieties are selected from reactive moieties that comprise electrophilic groups, reactive moieties that comprise nucleophilic groups, reactive moieties that comprise diene groups, reactive moieties that comprise dieneophile groups, reactive moieties that comprise alkenyl-containing groups, reactive moieties that comprise strained alkyne groups, and reactive moieties that comprise azide groups.

In some embodiments, which can be used in conjunction with the above aspects and embodiments, the iodine-containing core region comprises one or more iodinated aromatic groups.

In some embodiments, which can be used in conjunction with the above aspects and embodiments, the iodine-containing core region comprises a chain of two or more iodinated aromatic groups. For example, the iodinated aromatic groups may be directly linked together or may be linked together by a linking moiety that comprises an ether group, an ester group, an amide group, an amine group, a carbonate group.

In some embodiments, which can be used in conjunction with the above aspects and embodiments, the iodine-containing core region comprises two or more iodinated aromatic groups, the two or more iodinated aromatic groups each being linked to a central moiety though amide linkages. For example, the two or more iodinated aromatic groups may each be linked to a residue of a polyamine compound through an amide linkage or the two or more iodinated aromatic groups may each be linked to a residue of a polyacid compound through an amide linkage.

In some embodiments, which can be used in conjunction with the above aspects and embodiments, wherein the iodinated aromatic groups are iodine-substituted monocyclic aromatic groups or iodine-substituted multicyclic aromatic groups.

In some embodiments, which can be used in conjunction with the above aspects and embodiments, the iodine-containing core region comprises a residue of an iodinated polyhydroxylated initiator having a plurality of hydroxyl groups and the plurality of polymer arms correspond in number the number of hydroxyl groups in the iodinated polyhydroxylated initiator.

In some aspects, the present disclosure provides methods of forming a radiopaque, reactive multi-arm polymer in accordance with any of the above aspects and embodiments, the methods comprising conducting a ring-opening polymerization reaction in the presence of an iodinated polyhydroxylated initiator having a plurality of hydroxyl groups to form one of the plurality of polymer segments at each of the hydroxyl groups.

In some aspects, the present disclosure provides a system for forming a hydrogel composition that comprises (a) a radiopaque, reactive multi-arm polymer in accordance with any of the above aspects and embodiments and (b) multifunctional crosslinking compound comprising a plurality of complementary reactive moieties that are reactive with the reactive moieties of the radiopaque, reactive multi-arm polymer.

In some embodiments, the system comprises a first composition that comprises the radiopaque, reactive multi-arm polymer and a second composition that comprises the multifunctional crosslinking compound.

In some embodiments, the system comprises a first composition that comprises the radiopaque, reactive multi-arm polymer and the multifunctional crosslinking compound and a second composition that comprises an accelerant.

In some embodiments, which can be used in conjunction with the above aspects and embodiments, the system comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the system further comprises a delivery device.

In some aspects, the present disclosure provides a crosslinked reaction product of (a) a radiopaque, reactive multi-arm polymer in accordance with any of the above aspects and embodiments and (b) multifunctional crosslinking compound comprising a plurality of complementary reactive moieties that are reactive with the reactive moieties of the radiopaque, reactive multi-arm polymer.

In some embodiments, the crosslinked reaction product is in the form of particles.

In some aspects, the present disclosure provides a method of treatment comprising administering to a subject a mixture that comprises (a) a radiopaque, reactive multi-arm polymer in accordance with any of the above aspects and embodiments and (b) a multifunctional crosslinking compound having a plurality of complementary reactive moieties that are reactive with the reactive moieties of the radiopaque, reactive multi-arm polymer, wherein the mixture is administered under conditions such that the radiopaque, reactive multi-arm polymer and the multifunctional crosslinking compound crosslink in the subject after administration.

Potential benefits associated with the present disclosure include one or more of the following: radiocontrast is tunable, in vivo persistence is obtained, and a wide range of polymer arms can be provided.

The above and other aspects, embodiments, features and benefits of the present disclosure will be readily apparent from the following detailed description.

### BRIEF DESCRIPTION OF THE DRAWINGS

FIG. 1 schematically illustrates a method of forming an iodinated polyhydroxylated initiator, in accordance with an embodiment of the present disclosure.

FIG. 2 schematically illustrates a method of forming an iodinated polyhydroxylated initiator, in accordance with another embodiment of the present disclosure.

FIG. 3 schematically illustrates a method of forming an iodinated polyhydroxylated initiator, in accordance with another embodiment of the present disclosure.

FIG. 4 schematically illustrates a method of forming an iodinated polyhydroxylated initiator, in accordance with another embodiment of the present disclosure.

FIG. 5 schematically illustrates a method of forming an iodinated polyhydroxylated initiator, in accordance with another embodiment of the present disclosure.

FIG. 6 schematically illustrates methods of polymerizing radiopaque, multi-arm polymers using an iodinated polyhydroxylated initiator, in accordance with two embodiments of the present disclosure.

FIG. 7 schematically illustrates methods of polymerizing radiopaque, multi-arm polymers using an iodinated polyhydroxylated initiator, in accordance with two further embodiments of the present disclosure.

FIG. 8 illustrates a delivery device, in accordance with an embodiment of the present disclosure.

FIG. 9 illustrates a delivery device, in accordance with another embodiment of the present disclosure.

### DETAILED DESCRIPTION

The present disclosure pertains to radiopaque, reactive multi-arm polymers. As discussed below, the radiopaque, reactive multi-arm polymers have inherent radiopacity without the need to provide iodinated species onto ends of polymer arms, thus avoiding loss of reactive groups.

Radiopaque, reactive multi-arm polymers in accordance with the present disclosure include polymers that comprise a plurality of polymer arms linked to an iodine-containing core region. The radiopaque, reactive multi-arm polymers may have two, three, four, five, six, seven, eight, nine, ten, fifteen, twenty, twenty-five or more polymer arms.

As discussed in more detail below, in some embodiments, the iodine-containing core region comprises a residue of an iodinated multi-functional initiator, specifically, a residue of an iodinated polyhydroxylated initiator having two, three, four, five, six, seven, eight, nine, ten, fifteen, twenty, twenty-five or more hydroxyl groups, with the number of polymer arms corresponding to the number of functional groups, for example, the number of hydroxyl groups, in the initiator that is used to form the radiopaque, reactive multi-arm polymer.

The polymer arms of the radiopaque, reactive multi-arm polymers may each comprise a polymer segment linked to the iodine-containing core region and a terminal reactive moiety.

In some embodiments, a hydrolysable ester group is provided between the reactive moiety and the polymer segment. In some of these embodiments, the polymer arms of the radiopaque, reactive multi-arm polymers each comprise a polymer segment linked to the iodine-containing core region, a cyclic anhydride residue that is covalently linked to the polymer segment, and a reactive moiety that is covalently linked to the cyclic anhydride residue.

Reactive moieties may be selected from reactive moieties that comprise electrophilic groups, reactive moieties that comprise nucleophilic groups, reactive moieties that comprise diene groups, reactive moieties that comprise dieneophile groups, reactive moieties that comprise alkenyl-containing groups, reactive moieties that comprise strained alkyne groups, reactive moieties that comprise azide groups, ketone groups. aldehyde groups or acrylate groups, among others.

Reactive electrophilic groups include cyclic imide ester groups, such as succinimide ester groups,

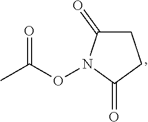

maleimide ester groups, glutarimide ester groups, diglycolimide ester groups, phthalimide ester groups, and bicyclo[2.2.1]hept-5-ene-2,3-dicarboxylic acid imide ester groups,

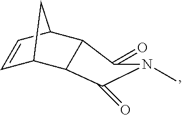

imidazole ester groups, imidazole carboxylate groups and benzotriazole ester groups, among other possibilities. Reactive nucleophilic groups include primary amine groups, thiol groups and hydroxyl groups, among other possibilities. Reactive diene containing groups include furan groups and tetrazine groups. Reactive dieneophile groups include norbornene groups and maleimide groups. Reactive alkenyl-containing groups include vinyl groups, acryloyl groups, a methacryloyl groups and strained alkene groups, for example, from cyclooct-4-en-1-yl groups,

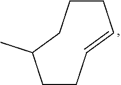

among other possibilities. Reactive strained alkyne groups include (1R,8S,9s)-bicyclo[6.1.0]non-4-yn-9-yl groups,

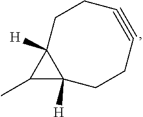

among other possibilities.

Polymer segments for the polymer arms can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the multi-arm polymers of the present disclosure typically contain between 10 and 1000 monomer or more units.

As previously noted, the radiopaque, reactive multi-arm polymers of the present disclosure have two or more polymer arms that extend from an iodine-containing core region. In various embodiments, the iodine-containing core region comprises a residue of an iodinated polyhydroxylated initiator having two, three, four, five, six, seven, eight, nine, ten, fifteen, twenty, twenty-five or more hydroxyl groups.

In certain embodiments, the iodine-containing core region comprises a residue of an iodinated polyhydroxylated initiator that comprises one or more iodinated aromatic groups. Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six or more iodine atoms. In various embodiments, the aromatic groups are further substituted with one or more hydroxyl-containing groups, which may be selected, for example, from hydroxyl groups and hydroxyalkyl groups (e.g., hydroxyalkyl groups containing one carbon, two carbons, three carbons, four carbons, etc.).

Specific examples of iodinated aromatic groups include those that comprise one or more monocyclic or multicyclic aromatic groups, substituted with (a) one or more iodine groups (e.g., one two, three, four, five, six or more iodine atoms) and (b) one or more hydroxyl-containing groups independently selected from one or more hydroxyl groups and one or more C₁-C₄-hydroxyalkyl groups, among others, which C₁-C₄-hydroxyalkyl groups may be linked to the monocyclic or multicyclic aromatic groups directly or through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups.

In some embodiments, the core region comprises multiple iodinated aromatic groups, for example, 2, 3, 4, 5, 6, 7, 8, 9, 10, or more aromatic groups, which may be linked together through any suitable linking moiety.

For example, multiple iodinated aromatic groups may be directly linked together.

As another example, multiple iodinated aromatic groups may be linked to one another through a suitable linking moiety, for example, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups.

As another example, multiple iodinated aromatic groups may each be attached to a central moiety through a suitable linking moiety, for example, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups.

In some embodiments, the core region comprises a central moiety and multiple iodinated aromatic groups covalently linked to the central moiety.

In some of these embodiments, the radiopaque, reactive multi-arm polymer comprises a central moiety, a plurality of iodinated aromatic groups covalently linked to the central moiety, one or more polymer arms linked to the iodinated aromatic group, the polymer arms comprising a polymer segment, a terminal reactive moiety, and optionally, a cyclic anhydride residue positioned between the polymer segment and the terminal reactive moiety. For example, the central moiety may comprise, for example, a residue of a polyamine compound and the iodinated aromatic groups may be covalently linked to the central portion through an amide linkage. As another example, the central moiety may comprise, for example, a residue of a polyacid compound and the iodinated aromatic groups may be covalently linked to the central portion through an amide linkage.

For example, amide linkages may be formed in an amide coupling reaction wherein primary amine groups of a polyamine compound are reacted with a carboxyl group of a compound that comprises a monocyclic or multicyclic aromatic group that is substituted with a carboxyl-containing group, one or more iodines, and one or more hydroxyl-containing groups.

As another example, amide linkages may be formed in an amide coupling reaction wherein carboxyl groups of a polycarboxyl compound are reacted with a primary amine group of a compound that comprises a monocyclic or multicyclic aromatic group that is substituted with a primary-amine-containing group, one or more iodines, and one or more hydroxyl-containing groups.

Hydroxyl-containing groups may be independently selected, for example, from hydroxyl groups and C₁-C₄-hydroxyalkyl groups. Primary-amine-containing groups may be independently selected, for example, from amino groups and C₁-C₄-aminoalkyl groups. Carboxyl-containing groups may be independently selected, for example, from carboxyl groups and C₁-C₄-carboxyalkyl groups. C₁-C₄-hydroxyalkyl groups, C₁-C₄-aminoalkyl groups and C₁-C₄-carboxyalkyl groups may be linked to the monocyclic or multicyclic groups directly or through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups.

Several iodinated polyhydroxylated initiators will now be described.

Commercially available 1,3,5 triiodo-2,4,6-trishydroxymethylbenzene (CAS #178814-33-0),

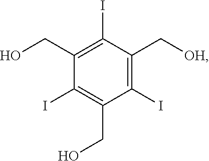 [SMILES: OCc1c(I)c(CO)c(I)c(CO)c1I]

can be used directly as an initiator for the polymerization of a 3-arm polymer with 3 iodine atoms at the core. Similarly, commercially available iodixanol (CAS #92339-11-2),

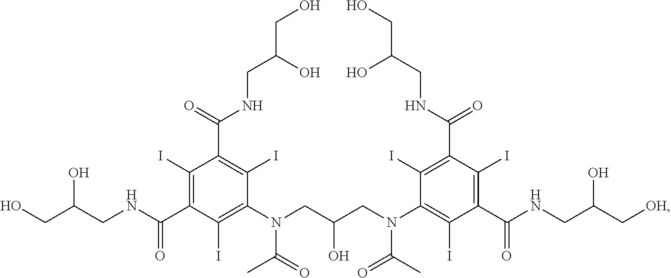 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

can be used directly as an initiator for the polymerization of an 8-arm polymer with 6 iodine atoms at the core.

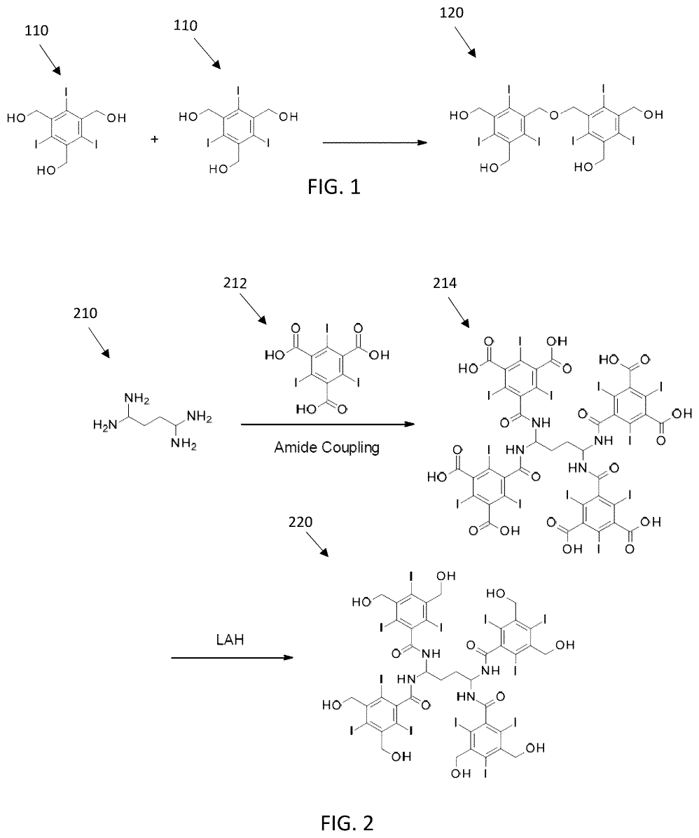

As another example, condensation of 1,3,5 triiodo-2,4,6-trishydroxymethylbenzene 110 can be carried out under acrolein and NaOH in water as shown in FIG. 1. Such a condensation reaction can be used to form iodinated polyhydroxylated initiators of varying length, allowing for tunability of the iodine content of the resulting multi-arm polymer. Examples include a dimer 120 as show in FIG. 1, which can be used as an initiator for the polymerization of a 4-arm polymer with 6 iodine atoms at the core, a trimer,

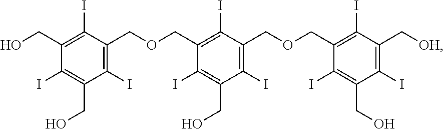 [SMILES: OCc1c(I)c(CO)c(I)c(COCc2c(I)c(CO)c(I)c(COCc3c(I)c(CO)c(I)c(CO)c3I)c2I)c1I]

which can be used as an initiator for the polymerization of a 5-arm polymer with 9 iodine atoms at the core, a tetramer,

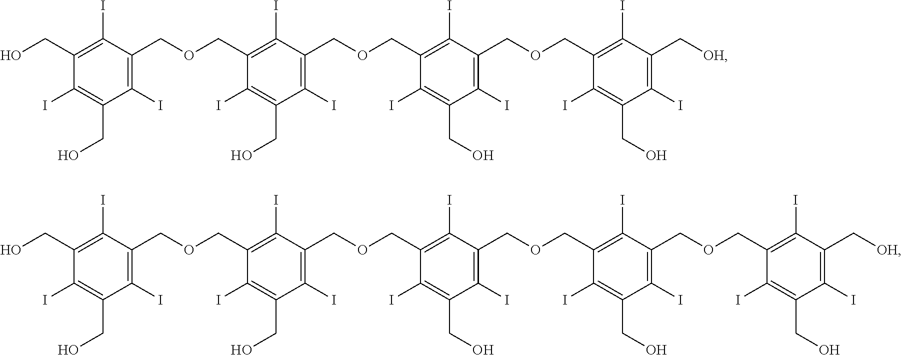 [SMILES: OCc1c(I)c(CO)c(I)c(COCc2c(I)c(CO)c(I)c(COCc3c(I)c(CO)c(I)c(COCc4c(I)c(CO)c(I)c(CO)c4I)c3I)c2I)c1I ; OCc1c(I)c(CO)c(I)c(COCc2c(I)c(CO)c(I)c(COCc3c(I)c(CO)c(I)c(COCc4c(I)c(CO)c(I)c(COCc5c(I)c(CO)c(I)c(CO)c5I)c4I)c3I)c2I)c1I]

a pentamer,
which can be used as an initiator for the polymerization of a 7-arm polymer with 15 iodine atoms at the core, a hexamer,

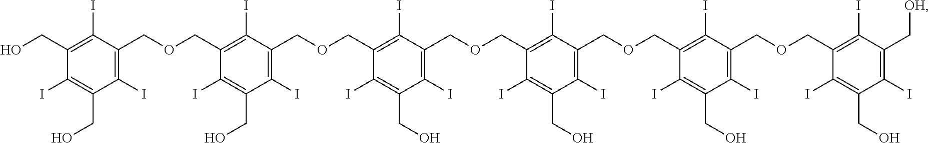 [SMILES: OCc1c(I)c(CO)c(I)c(COCc2c(I)c(CO)c(I)c(COCc3c(I)c(CO)c(I)c(COCc4c(I)c(CO)c(I)c(COCc5c(I)c(CO)c(I)c(COCc6c(I)c(CO)c(I)c(CO)c6I)c5I)c4I)c3I)c2I)c1I]

which can be used as an initiator for the polymerization of an 8-arm polymer with 18 iodine atoms at the core, and so forth.

With reference now to FIG. 2, 1,1,4,4-butanetetramine (CAS #2073104-72-8) 210 may be used as a starting material to couple with four 2,4,6-triiodo-1,3,5-benzenetricarboxylic acid molecules 212 in the presence of a carbodiimide coupling agent such as 1-ethyl-3-(3-dimethylaminopropyl) carbodiimide) (EDC), thereby forming amide bonds. The resulting iodinated polycarboxylated compound 214, comprises four monocyclic aromatic groups that are each substituted with two carboxy groups and three iodine groups, the four monocyclic aromatic groups linked to a central moiety (i.e., a tetramine residue) via four amide linkages. This compound 214 may then be treated with a reducing agent such as LiAlH₄ (LAH) to obtain an iodinated polyhydroxylated compound 220, which comprises four monocyclic aromatic groups that are each substituted with two hydroxyalkyl groups and three iodine groups, the four monocyclic aromatic groups linked to a central moiety (i.e., a tetramine residue) via four amide linkages. This compound 220 can be used as an initiator for the polymerization of an 8-arm polymer with 12 iodine atoms at the core.

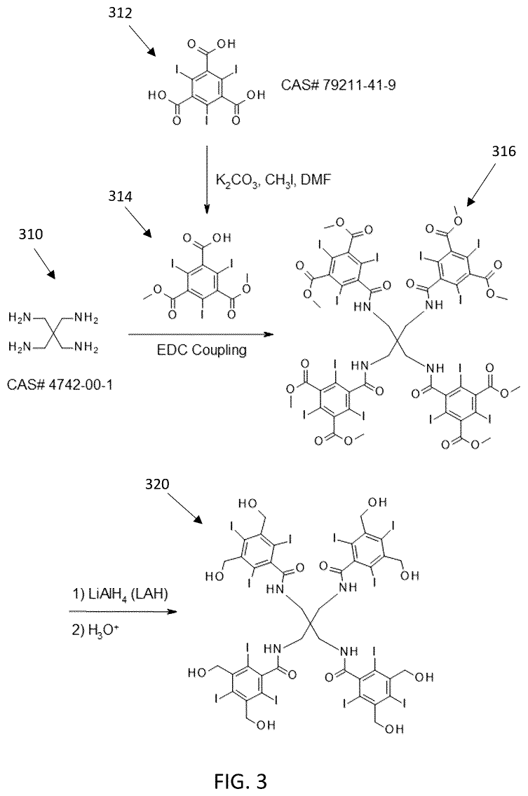

With reference now to FIG. 3, in another synthetic scheme, 2,4,6-triiodo-1,3,5-benzenetricarboxylic acid 312 is reacted with methyl iodide (CH₃I) in dimethylformamide (DMF) and potassium carbonate (K₂CO₃) to form 2,4,6-triiodo-1-carboxyl-3,5-methoxycarbonylbenzene 314. Pentaerythrityltetramine (CAS #4742-00-1) 310 is then reacted with the 2,4,6-triiodo-1-carboxyl-3,5-methoxycarbonylbenzene 314 in an amide coupling reaction in the presence of EDC, thereby forming four amide bonds. The resulting compound 316 comprises four monocyclic aromatic groups that are each substituted with two methoxycarbonyl groups and three iodine groups, the four monocyclic aromatic groups linked to a central moiety (i.e., a tetramine residue) via amide linkages. The compound 316 may then be treated with a reducing agent such as LiAlH₄ (LAH) and hydronium ion (H₃O⁺) to obtain an iodinated polyhydroxylated compound 320, which comprises four monocyclic aromatic groups that are each substituted with two hydroxyalkyl groups and three iodine groups, the four monocyclic aromatic groups linked to central moiety (i.e., a tetramine residue) via amide linkages. This compound 320 can be used as an initiator for the polymerization of an 8-arm polymer with 12 iodine atoms at the core.

In the above and other reactions, other polyamines may be employed beyond 1,1,4,4-butanetetramine and pentaerythrityltetramine, including 1,4,7,10-tetraazacyclododecane,

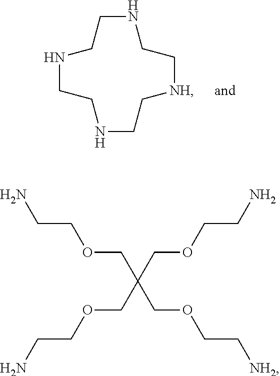 [SMILES: C1CNCCNCCNCCN1 ; NCCOCC(COCCN)(COCCN)COCCN]

among many others.

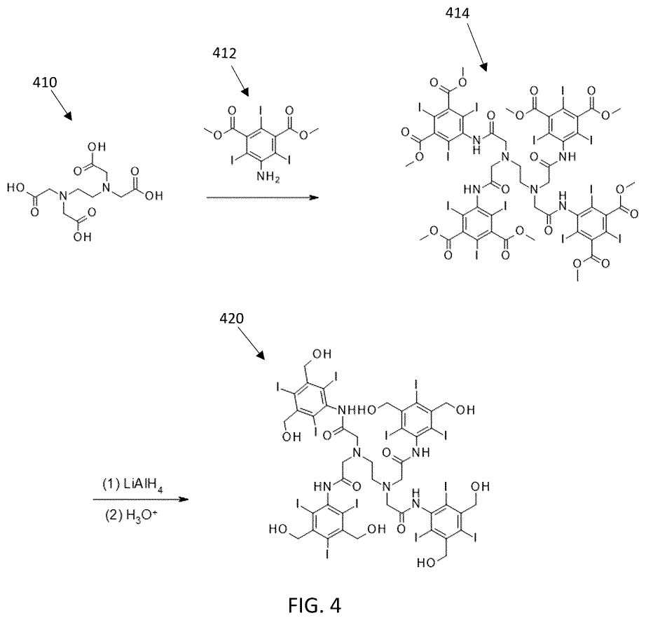

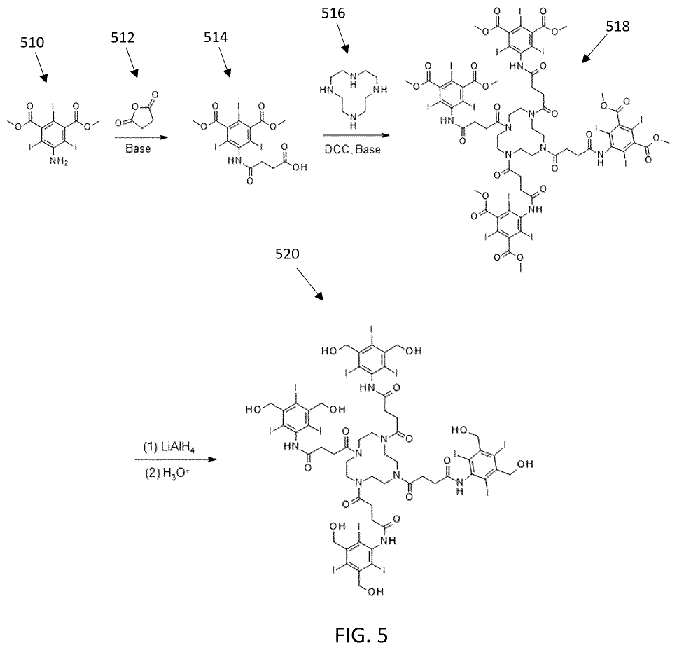

With reference now to FIG. 4, a polycarboxylic acid compound, specifically, 2-[2-[bis(carboxymethyl)amino]ethyl-(carboxymethyl)amino]acetic acid 410 may be used as a starting material to couple with four 2,4,6-triiodo-1-amino-3,5-methoxycarbonylbenzene molecules 412 in the presence of a carbodiimide coupling agent such as 1-ethyl-3-(3-dimethylaminopropyl) carbodiimide) (EDC), thereby forming amide bonds between the carboxy groups of compound 410 and the amino group of compound 412. The resulting compound 414 comprises four monocyclic aromatic groups that are each substituted with two methoxycarbonyl groups and three iodine groups, the four monocyclic aromatic groups being linked to central moiety (i.e., a tetracarboxylic acid residue) via amide linkages. The compound 414 may then be treated with a reducing agent such as LiAlH₄ (LAH) and hydronium (H₃O⁺) to obtain an iodinated polyhydroxylated compound 420, which comprises four monocyclic aromatic groups that are each substituted with two hydroxyalkyl groups and three iodine groups, the four monocyclic aromatic groups being linked to central moiety (i.e., a tetracarboxylic acid residue) via amide linkages. This compound 420 can be used as an initiator for the polymerization of an 8-arm polymer with 12 iodine atoms at the core.

With reference to FIG. 5, as an initial step, a 2,4,6-triiodo-1-carboxyethylamido-3,5-methoxycarbonylbenzene compound 514 is formed by reacting 2,4,6-triiodo-1-amino-3,5-methoxycarbonylbenzene 510 with succinic anhydride 512 under basic conditions. Then, four 2,4,6-triiodo-1-carboxyethylamido-3,5-methoxycarbonylbenzene molecules 514 are coupled with a polyamine compound 516, specifically, 1,4,7,10-tetraazacyclododecane, in the presence of a carbodiimide coupling agent such as 1-ethyl-3-(3-dimethylaminopropyl) carbodiimide) (EDC), thereby forming amide bonds between the carboxy group of compound 514 and the amino groups of compound 516. The resulting compound 518 comprises four monocyclic aromatic groups that are each substituted with two methoxycarbonyl groups and three iodine groups linked to central moiety (i.e., a tetraamine residue) via amide linkages. The compound 518 may then be treated with a reducing agent such as LiAlH₄ (LAH) and hydronium (H₃O⁺) to obtain an iodinated polyhydroxylated compound 520, which comprises four monocyclic aromatic groups that are each substituted with two hydroxyalkyl groups and three iodine groups linked to central moiety (i.e., a tetraamine residue) via amide linkages. This compound 520 can be used as an initiator for the polymerization of an 8-arm polymer with 12 iodine atoms at the core.

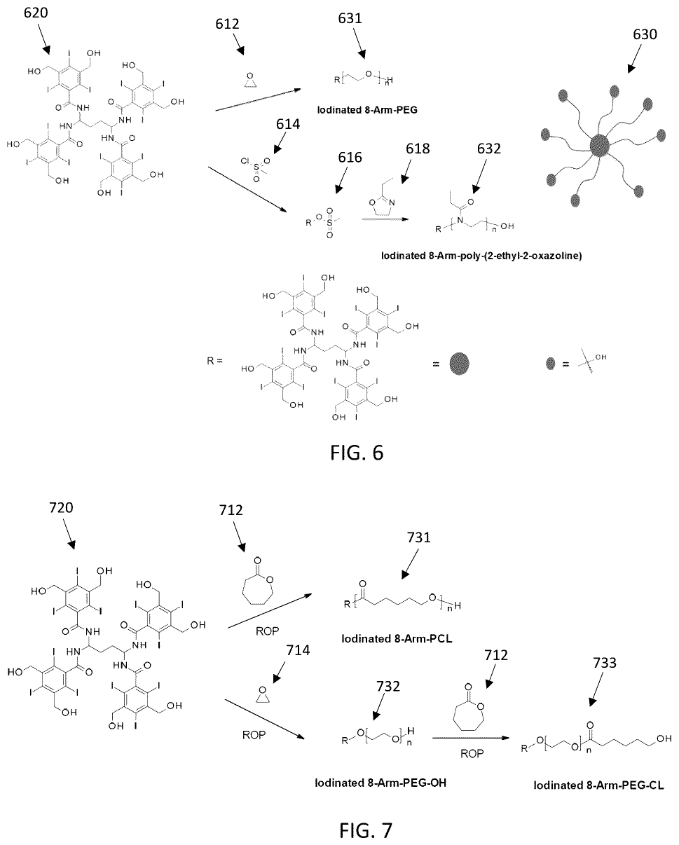

Iodinated polyhydroxylated initiators like those described above, among others, can be used to form radiopaque multi-arm polymers. For example, and with reference to FIG. 6, an iodinated polyhydroxylated initiator 620, specifically, the iodinated polyhydroxylated initiator described in conjunction with FIG. 2, may be used to initiate a ring-opening polymerization step with ethylene oxide 612 to form an iodinated, multi-arm polymer 631 having eight hydroxy-terminated poly(ethylene oxide) segments (only one of the eight hydroxy-terminated poly(ethylene oxide) segments is illustrated). The overall 8-arm-poly(ethylene oxide) is represented schematically by 630.

As also shown in FIG. 6, the iodinated polyhydroxylated initiator 620 can be reacted with a sulfonic acid chloride, specifically, methanesulfonyl chloride 614, to form a multifunctional initiator molecule 616 comprising a plurality of sulfonyl polymerization groups 616 (only one of the eight methanesulfonyl groups is illustrated). Other sulfonic acid chlorides, such as trifluoromethanesulfonyl chloride,

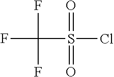

or tosyl chloride

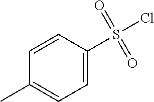

may be employed as well. The multifunctional initiator molecule 616 is then used to create a radiopaque, multi-arm polyoxazoline via ring opening polymerization of one or more oxazoline monomers, specifically 2-ethyl-2-oxazoline 618 with the formation of an iodinated, multi-arm polymer 632 having eight hydroxy-terminated poly(2-ethyl-2-oxazoline) segments (only one of the eight hydroxy-terminated poly(2-ethyl-2-oxazoline) segments is illustrated).

As another example, and with reference to FIG. 7, an iodinated polyhydroxylated initiator 720, specifically, the iodinated polyhydroxylated initiator of FIG. 2, is used to initiate a ring-opening polymerization step with ε-caprolactone 712 to form an iodinated, multi-arm polymer 731 having eight hydroxy-terminated poly(ε-caprolactone) segments (only one of the eight hydroxy-terminated poly(ε-caprolactone) segments is illustrated).

As also shown in FIG. 7, the iodinated polyhydroxylated initiator 720 is used to initiate a ring-opening polymerization step with ethylene oxide 714 to form an iodinated, hydroxy-terminated 8-arm-poly(ethylene oxide) 732. The iodinated, hydroxy-terminated 8-arm-poly(ethylene oxide) 732 is then used to initiate a ring-opening polymerization step with ε-caprolactone 712 to form an iodinated, hydroxy-terminated polymer multi-arm block copolymer 733 with each polymer arm comprising a poly(ethylene oxide) segment and a poly(ε-caprolactone) segment. It is noted that only one of the eight hydroxy-terminated poly(ethylene oxide-b-ε-caprolactone) arms is illustrated.

The approach shown in FIGS. 6 and 7 is applicable to the formation of other hydroxy-terminated polymers, including hydroxy-terminated polymers having polymer segments besides poly(ethylene oxide), poly(2-ethyl-2-oxazoline), and poly(ε-caprolactone) segments, selected, for example, from those disclosed above.

Radiopaque, iodinated reactive multi-arm polymers in accordance with the present disclosure can be formed from hydroxy-terminated iodinated multi-arm precursor polymers having arms that have terminal hydroxyl end groups like those described above. In particular embodiments, terminal hydroxyl groups of the polymer segments are reacted with acyclic anhydride compound (e.g., glutaric anhydride, succinic anhydride, malonic anhydride, adipic anhydride, diglycolic anhydride, etc.) to form an acid-end-capped polymer segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxy-terminated iodinated multi-arm hydrophilic precursor polymer under basic conditions to form a carboxylic-acid-terminated iodinated precursor polymer comprising a carboxylic acid end group that is linked to a polymer segment through a hydrolysable ester group.

A reactive moiety may then be linked to the carboxylic-acid-terminated iodinated precursor polymer. For example, an N-hydroxy cyclic imide compound (e.g., N-hydroxysuccinimide, N-hydroxymaleimide, N-hydroxyglutarimide, N-hydroxyphthalimide, N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated iodinated precursor polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester group (e.g., an succinimide ester group, an maleimide ester group, an glutarimide ester group, an phthalimide ester group, bicyclo[2.2.1]hept-5-ene-2,3-dicarboxylic acid imide ester group, etc.) that is linked to a polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

It should be noted that although iodine atoms are described above, other radiopaque atoms can be employed in place of the iodine atoms, including bromine.

In some aspects, the present disclosure provides a radiopaque hydrogel that comprises a crosslinked reaction product of (a) a radiopaque, reactive multi-arm polymer as described herein and (b) a multifunctional crosslinking compound having a plurality of complementary reactive moieties that are reactive with the reactive moieties of the radiopaque, reactive multi-arm polymer.

For example, a reaction product can be formed from the following: a radiopaque, reactive multi-arm polymer having electrophilic groups and a multifunctional crosslinking compound having nucleophilic groups; a radiopaque, reactive multi-arm polymer having nucleophilic groups and a multifunctional crosslinking compound having electrophilic groups; a radiopaque, reactive multi-arm polymer having diene groups and a multifunctional crosslinking compound having dienophilic groups; a radiopaque, reactive multi-arm polymer having dienophilic groups and a multifunctional crosslinking compound having diene groups; a radiopaque, reactive multi-arm polymer having strained alkyne groups and a multifunctional crosslinking compound having azide groups; a radiopaque, reactive multi-arm polymer having azide groups and a multifunctional crosslinking compound having strained alkyne groups; a radiopaque, reactive multi-arm polymer having strained alkene groups and a multifunctional crosslinking compound having tetrazine groups; a radiopaque, reactive multi-arm polymer having tetrazine groups and a multifunctional crosslinking compound having strained alkene groups; a radiopaque, reactive multi-arm polymer having alkene groups and a multifunctional crosslinking compound having thiol groups; and a radiopaque, reactive multi-arm polymer having thiol groups and a multifunctional crosslinking compound having alkene groups.

In some aspects of the present disclosure, systems are provided that are configured to deliver (a) radiopaque, reactive multi-arm polymer as described herein and (b) a multifunctional crosslinking compound as described herein. The radiopaque, reactive multi-arm polymer and multifunctional crosslinking compound are comingled under conditions such that reactive groups of the radiopaque, reactive multi-arm polymer react and form covalent bonds with the complementary reactive groups of the multifunctional crosslinking compound. Such systems can be used to form radiopaque crosslinked hydrogels, either in vivo or ex vivo.

In some aspects of the present disclosure, systems are provided that comprise (a) a first composition that comprises a radiopaque, reactive multi-arm polymer as described herein and (b) a second composition that comprises a multifunctional crosslinking compound as described herein. For example, the first and second compositions can be first and second fluid compositions that, when the first and second fluid compositions are mixed, covalent bonds form between the radiopaque, reactive multi-arm polymer and the multifunctional crosslinking compound, resulting in a crosslinked reaction product of the radiopaque, reactive multi-arm polymer and the multifunctional crosslinking compound.

In some embodiments, systems are provided that comprise (a) a first composition containing the radiopaque, reactive multi-arm polymer and the multifunctional crosslinking compound and (b) a second composition comprising an accelerant that accelerates a crosslinking reaction between the radiopaque, reactive multi-arm polymer and the multifunctional crosslinking compound. For example, the first composition may be a fluid composition in which the radiopaque, reactive multi-arm polymer and the multifunctional crosslinking compound are intermixed under conditions where crosslinking is suppressed between the reactive moieties of the radiopaque, reactive multi-arm polymer and the complementary reactive groups of the multifunctional crosslinking compound, and the second composition may be a fluid composition that, when mixed with the first fluid composition, causes covalent bonds to form between the radiopaque, reactive multi-arm polymer and the multifunctional crosslinking compound, resulting in a crosslinked reaction product of the radiopaque, reactive multi-arm polymer and the multifunctional crosslinking compound. In certain embodiments, the accelerant in the second fluid composition changes the pH of the first fluid composition, resulting in crosslinking between the radiopaque, reactive multi-arm polymer and the multifunctional crosslinking compound.

The first composition may be a first fluid composition or may be first dry composition to which a suitable fluid such as water for injection, saline, etc. can be added to form a first fluid composition. The second composition may independently be a second fluid composition or may be second dry composition to which a suitable fluid such as water for injection, saline, etc. can be added to form a second fluid composition. The first and second compositions may independently be provided in vials, syringes, or other reservoirs.

The first and second compositions may further comprise additional agents, including therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second fluid compositions to a subject. Preferred subjects include mammalian subjects, particularly human subjects.

In some embodiments, the system may include a delivery device that comprises a first reservoir that contains a first fluid composition that comprises a radiopaque, reactive multi-arm polymer as described above and a second reservoir that contains a second fluid composition that comprises a multifunctional crosslinking compound as described above. When the first and second fluid compositions are mixed, crosslinking occurs between the radiopaque, reactive multi-arm polymer and the multifunctional crosslinking compound.

In some embodiments, the system may include a delivery device that comprises a first reservoir that contains a first fluid composition that comprises the radiopaque, reactive multi-arm polymer and the multifunctional crosslinking compound, and a second reservoir that contains a second fluid composition that is an accelerant composition. The second fluid composition, when mixed with the first fluid composition, results in crosslinking between the radiopaque, reactive multi-arm polymer and the multifunctional crosslinking compound.

In either case, during operation, the first fluid composition and second fluid composition are dispensed from the first and second reservoirs and combined, whereupon the multifunctional crosslinking compound and the radiopaque, reactive multi-arm polymer and crosslink with one another to form a radiopaque crosslinked hydrogel.

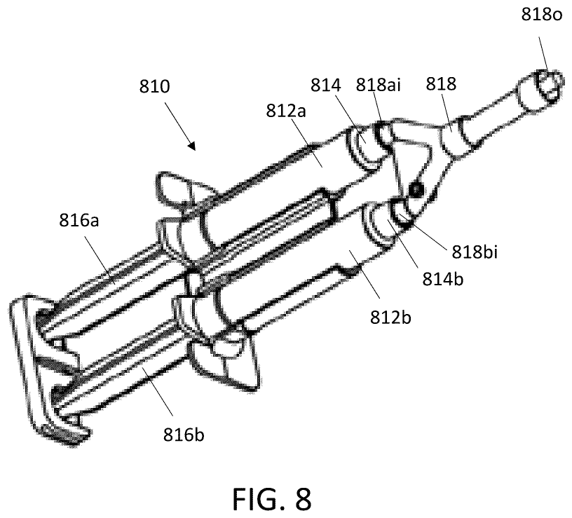

In particular embodiments, and with reference to FIG. 8, the system may include a delivery device 810 that comprises a double-barrel syringe, which includes first barrel 812a having a first barrel outlet 814a, which first barrel contains the first fluid composition, a first plunger 816a that is movable in the first barrel 812a, a second barrel 812b having a second barrel outlet 814b, which second barrel 812b contains the second fluid composition, and a second plunger 816b that is movable in the second barrel 812b. In some embodiments, the device 810 may further comprise a mixing section 818 having a first mixing section inlet 818ai in fluid communication with the first barrel outlet 814a, a second mixing section inlet 818bi in fluid communication with the second barrel outlet, and a mixing section outlet 818o.

In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a radiopaque crosslinked hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions or fluid admixtures of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, radiopaque crosslinked hydrogels may be in any desired form, including a slab, a cylinder, a coating, or a particle. In some embodiments, the radiopaque crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Radiopaque crosslinked hydrogel particles formed using the above and other techniques may varying widely in size, for example, having an average size ranging from 50 to 950 microns.

In addition to a radiopaque crosslinked hydrogel as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

Radiopaque crosslinked hydrogel compositions in accordance with the present disclosure include injectable fluid suspensions of radiopaque crosslinked hydrogel particles.

In various embodiments, kits are provided that include one or more delivery devices for delivering the radiopaque crosslinked hydrogel to a subject. Such systems may include one or more of the following: a syringe barrel, which may or may not contain a radiopaque crosslinked hydrogel as described herein; a vial, which may or may not contain a radiopaque crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked hydrogel may be provided in dry form (e.g., powder form) or in a form that is ready for injection, such as an injectable hydrogel form (e.g., a suspension of radiopaque crosslinked hydrogel particles).

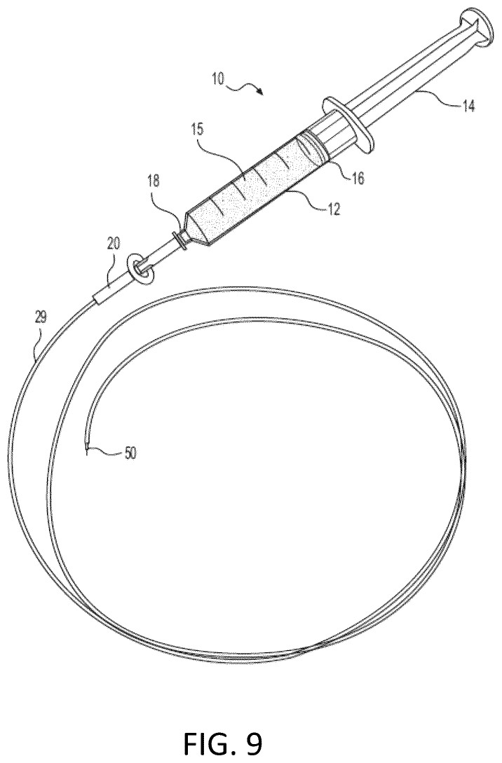

FIG. 9 illustrates a syringe 10 providing a reservoir for a radiopaque crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a radiopaque crosslinked hydrogel composition 15 for injection through the needle 50.

The radiopaque crosslinked hydrogel compositions described herein can be used for a number of purposes.

For example, radiopaque crosslinked hydrogel compositions can be injected to provide spacing between tissues, radiopaque crosslinked hydrogel compositions can be injected (e.g., in the form of blebs) to provide fiducial markers, radiopaque crosslinked hydrogel compositions can be injected for tissue augmentation or regeneration, radiopaque crosslinked hydrogel compositions can be injected as a filler or replacement for soft tissue, radiopaque crosslinked hydrogel compositions can be injected to provide mechanical support for compromised tissue, radiopaque crosslinked hydrogel compositions be injected as a scaffold, and/or radiopaque crosslinked hydrogel compositions can be injected as a carrier of therapeutic agents in the treatment of diseases and cancers and the repair and regeneration of tissue, among other uses.

The radiopaque crosslinked hydrogel compositions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The radiopaque crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

After administration, the radiopaque crosslinked hydrogel compositions of the present disclosure can be imaged using a suitable imaging technique.

Radiopaque crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A radiopaque, reactive multi-arm polymer comprising an iodine-containing core region, a plurality of polymer arms comprising a plurality of polymer segments linked to the iodine-containing core region, and a plurality of reactive moieties linked to the plurality of polymer segments.

2. The radiopaque, reactive multi-arm polymer of claim 1, further comprising a plurality of hydrolysable ester groups positioned between the plurality of reactive moieties and the plurality of polymer segments.

3. The radiopaque, reactive multi-arm polymer of claim 1, wherein the plurality of polymer segments comprise one or more monomer residues selected from C₁-C₄-alkylene oxide residues, oxazoline monomer residues, cyclic ester monomer residues and propylene oxide and fumarate comonomers (that can generate degradable copolymer).

4. The radiopaque, reactive multi-arm polymer of claim 1, wherein the polymer segments contain between 10 and 1000 monomer residues.

5. The radiopaque, reactive multi-arm polymer of claim 1, wherein the reactive moieties are selected from reactive moieties that comprise electrophilic groups, reactive moieties that comprise nucleophilic groups, reactive moieties that comprise diene groups, reactive moieties that comprise dieneophile groups, reactive moieties that comprise alkenyl-containing groups, reactive moieties that comprise strained alkyne groups, and reactive moieties that comprise azide groups.

6. The radiopaque, reactive multi-arm polymer of claim 1, wherein the iodine-containing core region comprises one or more iodinated aromatic groups.

7. The radiopaque, reactive multi-arm polymer of claim 1, wherein the iodine-containing core region comprises a chain of two or more iodinated aromatic groups.

8. The radiopaque, reactive multi-arm polymer of claim 7, wherein the iodinated aromatic groups are directly linked together or linked together by a linking moiety that comprises an ether group, an ester group, an amide group, an amine group, a carbonate group.

9. The radiopaque, reactive multi-arm polymer of claim 1, wherein the iodine-containing core region comprises two or more iodinated aromatic groups, the two or more iodinated aromatic groups each being linked to a central moiety though amide linkages.

10. The radiopaque, reactive multi-arm polymer of claim 9, wherein the two or more iodinated aromatic groups are each linked to a residue of a polyamine compound through an amide linkage or wherein the two or more iodinated aromatic groups are each linked to a residue of a polyacid compound through an amide linkage.

11. The radiopaque, reactive multi-arm polymer of claim 1, wherein the iodinated aromatic groups are iodine-substituted monocyclic aromatic groups or iodine-substituted multicyclic aromatic groups.

12. The radiopaque, reactive multi-arm polymer of claim 1, wherein the iodine-containing core region comprises a residue of an iodinated polyhydroxylated initiator having a plurality of hydroxyl groups and wherein the plurality of polymer arms correspond in number the number of hydroxyl groups in the iodinated polyhydroxylated initiator.

13. A method of forming the radiopaque, reactive multi-arm polymer of claim 1, comprising conducting a ring-opening polymerization reaction in the presence of the iodinated polyhydroxylated initiator to form one of the plurality of polymer segments at each of the hydroxyl groups.

14. A system for forming a hydrogel composition that comprises (a) a radiopaque, reactive multi-arm polymer comprising an iodine-containing core region, a plurality of polymer arms comprising a plurality of polymer segments linked to the iodine-containing core region, and a plurality of reactive moieties linked to the plurality of polymer segments and (b) multifunctional crosslinking compound comprising a plurality of complementary reactive moieties that are reactive with the reactive moieties of the radiopaque, reactive multi-arm polymer.

15. The system of claim 14, wherein the system comprises a first composition that comprises the radiopaque, reactive multi-arm polymer and a second composition that comprises the multifunctional crosslinking compound.

16. The system of claim 14, wherein the system comprises a first composition that comprises the radiopaque, reactive multi-arm polymer and the multifunctional crosslinking compound and a second composition that comprises an accelerant.

17. The system of claim 14, wherein the system comprises one or more additional agents selected from therapeutic agents, imaging agents, colorants, tonicity adjusting agents, suspension agents, wetting agents, and pH adjusting agents.

18. The system of claim 14, further comprising a delivery device.

19. A crosslinked reaction product of (a) a radiopaque, reactive multi-arm polymer in accordance with claim 1 and (b) multifunctional crosslinking compound comprising a plurality of complementary reactive moieties that are reactive with the reactive moieties of the radiopaque, reactive multi-arm polymer.

20. A method of treatment comprising administering to a subject a mixture that comprises (a) a radiopaque, reactive multi-arm polymer comprising an iodine-containing core region, a plurality of polymer arms comprising a plurality of polymer segments linked to the iodine-containing core region, and a plurality of reactive moieties linked to the plurality of polymer segments and (b) a multifunctional crosslinking compound having a plurality of complementary reactive moieties that are reactive with the reactive moieties of the radiopaque, reactive multi-arm polymer, wherein the mixture is administered under conditions such that the radiopaque, reactive multi-arm polymer and the multifunctional crosslinking compound crosslink in the subject after administration.
